(19) United States
(12) Patent Application Publication
(10) Pub. No.: US 2019/0231263 A1
(43) Pub. Date: Aug. 1, 2019

(54) MICRO SENSOR

(71) Applicant: DEBIOTECH S.A., Lausanne (CH)

(72) Inventors: Federico RIBET, Stockholm (SE); Göran STEMME, Lidingö (SE); Niclas ROXHED, Bromma (SE); Paul VESCOVO, Lausanne (CH); Francois CANNEHAN, Lausanne (CH)

(21) Appl. No.: 16/377,307

(22) Filed: Apr. 8, 2019

Related U.S. Application Data
(63) Continuation of application No. PCT/IB2017/056185, filed on Oct. 6, 2017.

(30) Foreign Application Priority Data
Oct. 7, 2016 (EP) 16192715.7

(51) Int. Cl. A61B 5/00; A61B 5/145; A61B 5/1486; A61L 31/08

(52) U.S. Cl. CPC A61B 5/685; A61B 5/14514; A61B 5/14532; A61B 2562/125; A61L 31/088; A61B 2562/028; A61B 5/14865

(57) ABSTRACT

A micro sensor for sensing at least one analyte from a biological fluid of a patient, the micro sensor comprises a probe and at least two electrodes arranged on the probe. The probe comprises a longitudinal body and the at least two electrodes are arranged in the width or in the length of the longitudinal body of the probe.

### FIELD OF INVENTION

The present invention relates to a device adapted to sense analytes present in a body fluid of a patient, such as (but not limited to) blood glucose concentration for example in the dermis of the patient. The present invention further relates to a microneedle adapted to comprise such sensor and manufacturing process adapted to produce such devices.

### STATE OF THE ART

Diabetes mellitus (DM) is a set of metabolic disorders caused by either a faulty bodily response (T2DM) or an underproduction in the pancreas (T1DM) of insulin, which regulates the metabolism of carbohydrates and controls hyperglycemia. These diseases lead to unstable and dangerously high oscillations of the glucose level in the body. According to the International Diabetes Federation, diabetes is now affecting 387 million people, and was responsible for 4.9 million deaths worldwide in 2014. Although diabetes is not curable, proper diabetes management is essential to avoid numerous possible complications, both on a short and a long term period, such as hypoglycemia, cardiovascular diseases, neuropathy, retinal damage, nephropathy and amputations. Traditionally, patients perform self-monitoring of their glycaemia through capillary blood sampling by finger pricking. Even though the measurement results are very reliable, there are several drawbacks for the patient, such as pain, risk of infections, lesions, and sensory loss, combined with an incomplete temporal picture resulting from 4-5 daily data points. To limit risks and improve the treatment, research in the field of real-time glucose monitoring of interstitial fluid (IF) has gained much attention in the last two decades. In fact, continuous glucose monitoring (CGM) can significantly enhance the quality of the treatment, offering a complete picture of glycaemia evolution, while dramatically reducing the discomfort of regular finger pricks required by conventional methods.

To monitor glycaemia, electrochemical enzymatic glucose sensing currently remains the most reliable monitoring technique available. Several alternatives have been investigated, including enzyme-free detection principles, without being able to supplant the former standard enzymatic principle. Implantable continuous glucose monitoring systems based on electrochemical sensing have been available on the market for over a decade (e.g. products by Medtronic, Dexcom, and Abbott). These products consist of flexible strips implantable in the hypodermis, measuring the glucose concentration in the interstitial fluid. However, although more convenient than traditional devices, these products are not as reliable as blood measurements and cannot yet be considered neither non-invasive nor painless due to their large needle-based insertion mechanism. For example, the most recent product on the market consists of a sensor strip of 5×0.6 mm², inserted under the skin through a 6 mm long half-pipe hypodermic needle. Other smaller planar amperometric glucose sensors have been previously designed, but all with a total sensing portion always exceeding 0.4 mm² and with a length in the insertion direction greater than 3 mm.

The miniaturization of sensor is more challenging when the sensor is configured to sense a fluid in the dermal layer of the patient skin. Indeed, such sensors are so miniaturized that the sensors may be more fragile and it may be difficult to reach the dermal layer. The present invention further discloses some features in order to also overcome these challenges.

### GENERAL DESCRIPTION OF THE INVENTION

According to the present invention, several solutions are provided to overcome the drawbacks of conventional art.

A first aspect of the invention is directed to a micro sensor designed to sense at least one analyte from a biological fluid of a patient preferentially in the dermis of the user. The micro sensor may comprise a probe having a longitudinal body and at least two electrodes arranged on the probe. The at least two electrodes are adapted to be arranged in the width or in the length of the longitudinal body of the probe.

One aim of this work is to investigate the possibility of a further significant sensor miniaturization, not only to permit reduction of the invasiveness and the discomfort related to current CGM systems, but also to enable directly access the interstitial fluid (IF) of the dermal region in the skin (as shown in FIG. 1).

In particular, according to one of embodiments, the present document discloses a complete microfabricated sensor with electrodes geometrically arranged to potentially fit on a probe with a length in the insertion direction significantly shorter than 1 mm, representing the average depth of human forearm dermis, and narrower than 75 μm for the ease of insertion. In fact, it has been shown that optimal access to interstitial fluid is achieved in the dermal region. In this location interstitial fluid is more abundant than in the underlying subcutaneous tissue, in which, in addition, the inhomogeneity of the adipose tissue and the size of the adipocytes may also detrimentally affect the interstitial fluid solutes' concentration. Feasibility of glycaemia monitoring in the dermal interstitial fluid has been previously demonstrated by interstitial fluid extraction. Moreover, there are indications that glucose concentration in the dermal interstitial fluid best matches the amplitude and dynamics of blood and plasma glucose. Finally, the implant size plays a role in the extent of the foreign body reaction (FBR), which is detrimental for in-vivo measurements. In fact, the FBR increases proportionally to the initial tissue trauma during insertion, which relates to the degree of acute inflammation, and to the implant size, which is responsible for the development of the fibrous capsule.

This document describes a micro sensor which may be an implantable planar amperometric glucose sensor with the smallest electrode footprint area reported up to date, which, despite the decreased size (overall area of the less sensing portion of less than 0.04 mm²) and the related challenges, is able to selectively measure physiologically relevant glucose concentrations in-vitro, with a resolution and a sensitivity comparable to commercial CGM systems.

Preferentially, the electrodes have a footprint area allowing the electrodes to be placed in the dermis of the patient. And optionally the probe may comprise three electrodes.

According to one embodiment, the electrodes are arranged in the width of the probe and are spaced apart there between.

According to one embodiment, the three electrodes are arranged in the length of the probe and are spaced apart there between.

Preferentially, in both cases, the distance between each electrode is as small as possible.

A second aspect of the invention is directed to a manufacturing process of such micro sensor comprising a substrate on which three electrodes are manufactured, the manufacturing process may comprise the following steps:
Deposit at least one conducting layer defining a first electrode, a second electrode and a third electrode,
Deposit an iridium oxide layer on the third electrode, and
Deposit at least one membrane on at least one of the three electrodes.

A third aspect of the invention is directed to a sensing device adapted to sense at least one analyte from a biological fluid of a patient. The sensing device comprises:
an hollow microneedle including:
   a longitudinal body which extends from a base to a distal end,
   at least one internal wall defining an internal cavity, and
   an opening configured to access the cavity from the exterior of the microneedle; and
a sensor device arranged at least partially into the cavity

In order to protect the sensor and to prevent coring of the skin when the micro needle is inserted (and/or to prevent clogging of the opening), the microneedle may comprise a hat arranged at the distal end in order to cover at least partially the cavity or the sensor device. This hat may be made from the same material of the microneedle or from a dissolvable material.

The sensor device may comprise at least two electrodes arranged in the width or in the length of the sensor. In both cases, the at least two electrodes are arranged into the same microneedle, preferentially into the same cavity. Furthermore, the sensing device may comprise a single microneedle and/or a single sensor which may be a micro sensor as described above.

A fourth aspect of the invention is directed to a sensing device adapted to sense at least one analyte from a biological fluid of a patient, the sensing device comprises:
A microneedle having:
   a longitudinal body which extends from a base to a distal end, and
   a first side opening (for example a substantial side opening); and
a sensor arranged at least partially into the side opening.

Said first side opening is configured to improve the contact between a sensor arranged into the microneedle and the interstitial fluid.

Preferentially, the first side opening may extend along of a determined length of the longitudinal body such that the first side opening reaches a determined depth of the patient skin when the microneedle is inserted into the patient skin.

For example, the side opening may extend at least along 25% (for example more than 50% or 80% or 100%) of a length of the longitudinal body such that the side opening reaches a determined depth of the patient skin when the microneedle is inserted into the patient skin.

Optionally, at least a part of the sensor is arranged into the side opening and the side opening may extend at least along 25% (for example more than 50% or 80% or 100%) of a length of a sensing part of the sensor, such that the sensing part of the sensor contacts the interstitial fluid at a determined depth.

Even if, the document describes in detail a glucose sensor, the invention is not to be understood as limited to such sensor.

### LIST OF FIGURES

The present invention will be better understood at the light of the following detailed description which contains non-limiting examples illustrated by the following figures:

FIG. 1a shows a view of the different layers of the skin. In-scale cross-sectional drawing of the skin layers, depicting the possible use of a sensor (1) with the presented miniaturized sensing area inserted in the dermal space, compared to the sensing portion of a state-of-the-art sensor (2) (i.e. Abbott Freestyle™ Libre) having a much deeper penetration depth, operating in the subcutaneous tissue (or hypodermis).

FIG. 1b shows a first embodiment of the device (1), with the active area of the three-electrode sensor inserted in the dermal space.

FIG. 1c shows a second embodiment of the device (1).

FIG. 2 illustrates a cross-sectional representation of potential microfabrication steps Diagrams are not to scale.

FIGS. 3 (A) and (B) show some examples of sensor device. (A) Optical top-view picture of the complete chip, with contacts on the top part, and active area, functionalized with the three polymeric membranes, in the lower part, as used for the characterization. (B) SEM picture of a bare chip sensing area, in the middle, compared to Abbott's Freestyle™ Libre sensor, on the right, and its insertion needle, on the left. Inset: SEM micrograph of the three microelectrodes before the deposition of the membranes, where WE and CE are made of platinum and the RE is covered with electrochemically oxidized iridium.

FIG. 4 shows the measurement of the open circuit potential of an oxidized iridium reference electrode (total geometrical area 0.012 mm2) in a standard oxygen-containing PBS solution at 26° C., over a four-day period. Inset: OCP measurement of another integrated iridium oxide Q-RE immediately after CV, in a deaerated PBS solution, showing a different absolute OCP value.

FIG. 5: (A) Chrono-amperometric measurement in PBS at increasing glucose concentrations, +50 mg/dL per step, and demonstration of selectivity towards possible interfering substances, namely UA and AA, using the described microsensor and the on-chip integrated IrOx Q-RE, over an eight-hour time frame. (B) Measured current as a function of the glucose concentration, from measurement A, and a linear fit of the curve. The linear fit follows the equation y=0.084 [nA·mg⁻¹·dl]·x+0.074 [nA], having a linear correlation coefficient of 0.999 with the measurement data. A linear fit on the entire range can be instead expressed as y=0.624 [nA·mg⁻¹·dl]·x+2.369 [nA], having a linear correlation coefficient of 0.986 with the measurement data. A bias of +0.5 V with respect to the embedded IrOx Q-RE was applied during measurements. (C) Chronoamperometric characterization of the selectivity of the sensor, biased at +0.5 V with respect to the embedded IrOx Q-RE, at 34° C. in PBS solution. Aliquots at three increasing concentrations (low, high, and toxic) of AA and secondly UA have been added to the stirred solution, before adding glucose at a concentration of 100 mg/dL. Inset: graphical representation of the contribution of the different substances to the overall current, in which the current generated by low physiological levels of interferents can be considered as a minimum offset.

FIG. 6 shows an embodiment wherein the electrodes are arranged in the length, or vertically (RE: Reference Electrode, CE: Counter Electrode, WE: Working Electrode).

FIG. 7 shows an embodiment wherein the electrodes are arranged in the width, or horizontally.

FIGS. 8 A, B and C show an embodiment of a microneedle in which a sensor is arranged.

FIGS. 9 A and B show an embodiment of a microneedle which comprises through holes and a sensor is arranged.

FIGS. 10 A, B and C show an embodiment of a microneedle which comprises substantial side opening and a sensor is arranged.

FIG. 11 show different possibilities for the design of the through hole on the backside.

FIG. 12 shows a potential process flow for microneedle adapted to receive a sensor.

### LIST OF ELEMENTS

1 device of the invention
2 state-of-art sensor
3 epidermis
4 dermis (˜1 mm)
5 hypodermis or subcutaneous tissue (>5 mm)
6 microneedle
7 electrical contacts
8 wire
9 hat
10 sensor
11 tip
12 base
13 support
14 body
15 head
16 opening
17 contact
18 contour of the microneedle
19 through hole on backside
20 conductive paths
21 backside of microneedle
100 silicon
101 silicon dioxide
102 conducting layer (for example Pt)
103 insulation layer (for example Si3N4)
104 Ir/IrO2
105 functionalization layer (for example Gox-BSA-GA)
106 PU (optional)
107 Nafion (optional)
108 first electrode
109 second electrode
110 third electrode

### DETAILED DESCRIPTION OF THE INVENTION

In the following detailed description, reference is made to the accompanying drawings that form a part hereof, and in which are shown by way of illustration several embodiments of devices, systems and methods. It is to be understood that other embodiments are contemplated and may be made without departing from the scope or spirit of the present disclosure. The following detailed description, therefore, is not to be taken in a limiting sense.

All scientific and technical terms used herein have meanings commonly used in the art unless otherwise specified. The definitions provided herein are to facilitate understanding of certain terms used frequently herein and are not meant to limit the scope of the present disclosure.

As used in this specification and the appended claims, the singular forms “a”, “an”, and “the” encompass embodiments having plural referents, unless the content clearly dictates otherwise.

As used in this specification and the appended claims, any direction referred to herein, such as “top”, “bottom”, “left”, “right”, “upper”, “lower”, and other directions or orientations are described herein for clarity in reference to the figures and are not intended to be limiting of an actual device or system. Devices and systems described herein may be used in a number of directions and orientations.

As used herein, “have”, “having”, “include”, “including”, “comprise”, “comprising” or the like are used in their open ended sense, and generally mean “including, but not limited to.

As used in this specification and the appended claims, the term “or” is generally employed in its sense including “and/or” unless the content clearly dictates otherwise.

The term “proximal” as used herein, is a broad term and is used in its ordinary sense, including, without limitation, near to a point of reference such as an origin or a point of attachment, for example, from the surface of the skin or the support of the microneedle.

The term “distal” as used herein, is a broad term and is used in its ordinary sense, including, without limitation, spaced relatively far from a point of reference, such as an origin or a point of attachment, for example, from the surface of the skin or the support of the microneedle

The term “substantially” as used herein, is a broad term and is used in its ordinary sense, including, without limitation, being largely but not necessarily wholly that which is specified.

### Working Principle

The amperometric sensor is based on the selective transformation of glucose molecules through glucose oxidase (GOx) into hydrogen peroxide (H2O2). The concentration of H₂O₂ is then anodically detected at the surface of the working electrode (WE), when a bias of +0.6 V is applied, according to the following set of reactions:

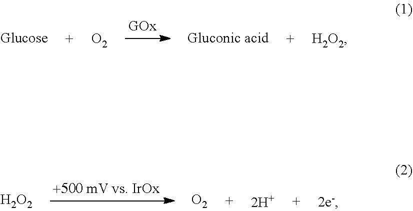

while at the counter electrode (CE) the current flow is balanced by the reduction reaction:

2H⁺+1/2O₂+2e⁻→H₂O (3).

Since oxygen is involved in reaction (1), an excess of O₂ with respect to glucose must be ensured at the WE surface in order to avoid undesirable saturation effects in the physiologically relevant glucose range. Moreover, at +0.6 V, other electroactive substances commonly present in the IF, such as uric acid and ascorbic acid, interfere with the detection of hydrogen peroxide, being undesirably oxidized, and contribute to the overall current, thus generating a false glucose reading. As a consequence, additional precautions are needed at the WE to ensure correct operation and selectivity of the sensor. Finally, in order to keep the WE potential fixed, and hence for the sensor to be stable and provide a reliable response over time, a third electrode, acting as reference, is necessary.

### Pseudo-Reference Electrode

When miniaturizing electrochemical components, the traditional macroscopic reference electrode configuration cannot be realized and a pseudo-reference electrode (Q-RE) must to be used. Integrating a reliable Q-RE represents a key challenge for microsensors. The standard material historically used as a reference electrode in these applications is silver-silver chloride (Ag/AgCl). Its use for microprobes has been shown through chlorination of thick electrodeposited silver layers and Cl-plasma treatment. Despite the well-known behavior of Ag/AgCl, when these electrodes are microfabricated they typically have a very short lifetime due to dissolution of the thin AgCl layer, and when the chloride coating is dissolved the standard potential radically changes. This behavior, in combination with concerns about toxicity of the AgCl solute and the related inflammatory response, makes Ag/AgCl a less than optimal candidate for long-term in-vivo measurements, especially in ultra-miniaturized devices. A promising alternative to overcome the described limitations is the use of an iridium oxide (IrOx) electrode, which has been previously demonstrated to be a good candidate for miniaturized pseudo-reference electrodes for in-vivo applications. In fact, IrOx shows good biocompatibility, mechanical stability and minimal potential drift. Despite its strong open circuit potential dependence on the pH of the solution (−77 mV/pH), IrOx is a reliable enough reference electrode in specific environments such as buffered solutions or interstitial fluid, where the pH variations are small and regulated in the range between 6.8 and 7.4. Furthermore, it shows minimal long-term potential drift, and allows for the use of thin films (sub-100 nm thickness) deposited by standard microfabrication technologies, followed by a simple one-step activation procedure in a phosphate buffered saline (PBS) solution. Finally, with respect to Ag/AgCl, IrOx shows much lower sensitivity to electroactive species present in the IF.

### Sensor Design

The present document discloses a sensor for sensing at least one analyte from a biological fluid of a patient (for example: a biochemical, metabolite, electrolyte, ion, pathogen or microorganism). The sensor comprises a probe having a longitudinal body and at least two electrodes arranged on the probe. Preferentially, the longitudinal body of the probe is designed in such manner that the at least two electrodes reaches a determined measurement area of the patient, for example a determined depth of a patient skin, such as the dermal layer of the patient skin. Thus, preferentially, the length of the probe is smaller than 1 mm, and the width of the probe may be smaller than 100 μm.

The sensor may comprise a non-conductive body on which the electrodes are arranged, electrically conductive paths connected to the electrodes and electrical contacts 17 (adapted to be connected to an electronics part (via wire for example) of the system which processes the data).

In one embodiment, the sensor may consist of a three-electrode configuration including a working electrode and a counter electrode which may be made of platinum, and an on-chip integrated pseudo-reference electrode which may be made of oxidized iridium.

The electrode size range may be between 1 and 1000 μm at least in one dimension, preferentially, between 100 and 400 μm at least in one dimension, more preferentially, between 30 and 70 μm at least in one dimension. The counter electrode (CE) may be twice as large as the working electrode (WE).

In particular, the range for each electrode may be:
WE 30×100 to 70×200 μm
CE 30×100 to 70×400 μm
RE 30×100 to 70×200 μm

The inter-electrode distance may be in the range 1 and 1000 μm, preferentially 5 and 50 μm. The distance between electrodes is minimal in order to maximize active electrode area which is crucial at this level of miniaturization. Furthermore, the miniaturization and the reduced inter-electrode distance allows for reduction in the noise level, and reduction of electrode polarization and ohmic losses (due to the ionic resistance of the skin which leads to increased overpotential and energy losses).

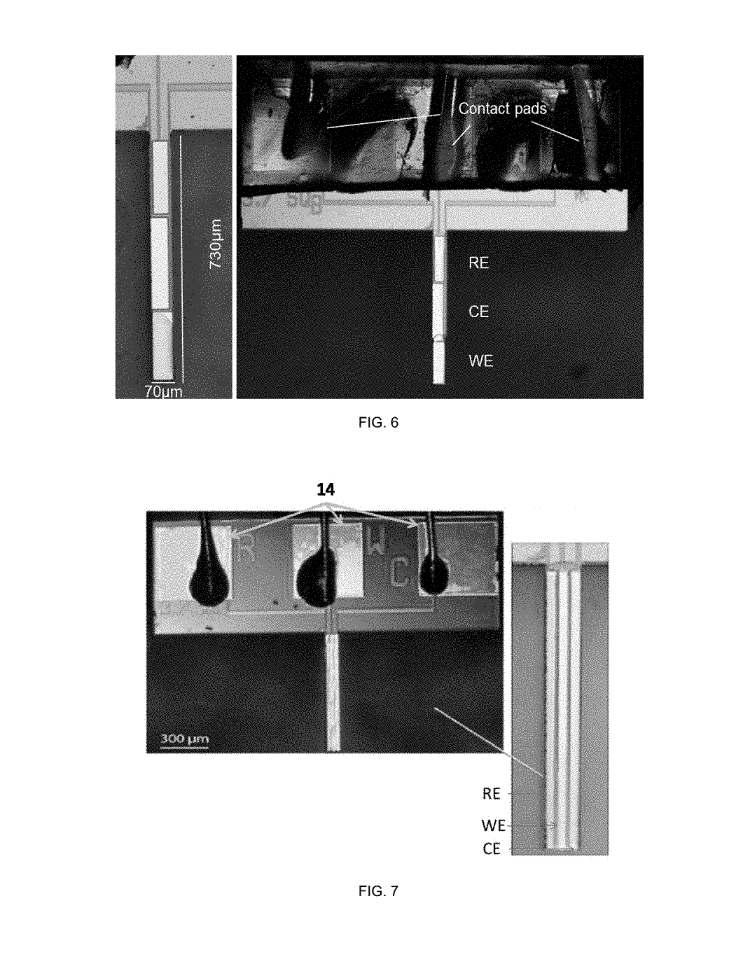

In one embodiment, the probe may be approximately 730 μm long, 70 μm wide and 70 μm thick. It may be narrower and thinner but preferentially not larger and more preferentially not shorter. In this case, the three electrodes are preferentially arranged vertically (in the length) (as shown by the FIG. 6), and this configuration allows for different coating deposition on the different electrodes. One of or each of the three electrodes may have a surface area of 0.012 mm² (170×70 μm²), with an inter-electrode distance of 10 μm, in order to maximize the active surface on the desired total sensor area and, at the same time, minimize ohmic losses. The resulting total sensing portion footprint is 0.037 mm² (530×70 μm²).

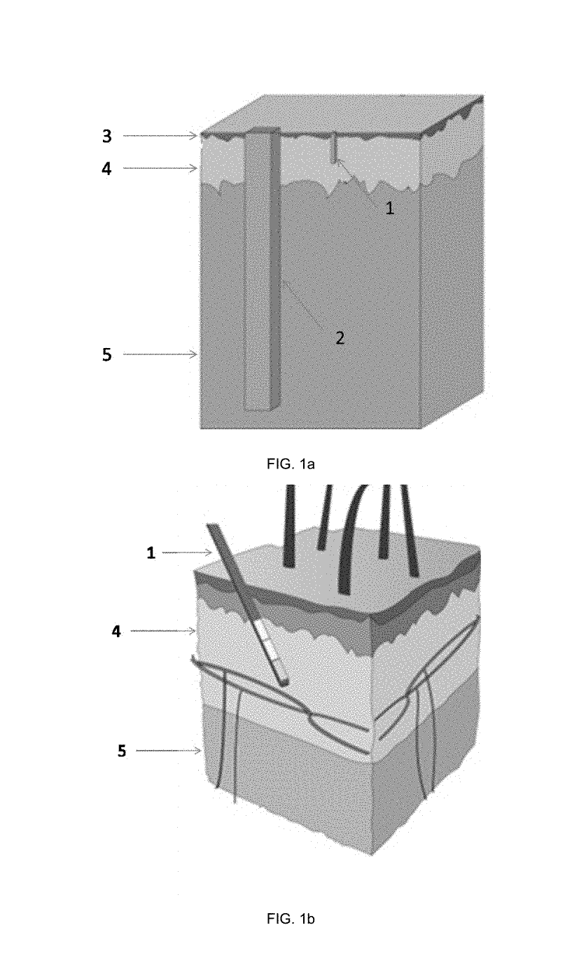

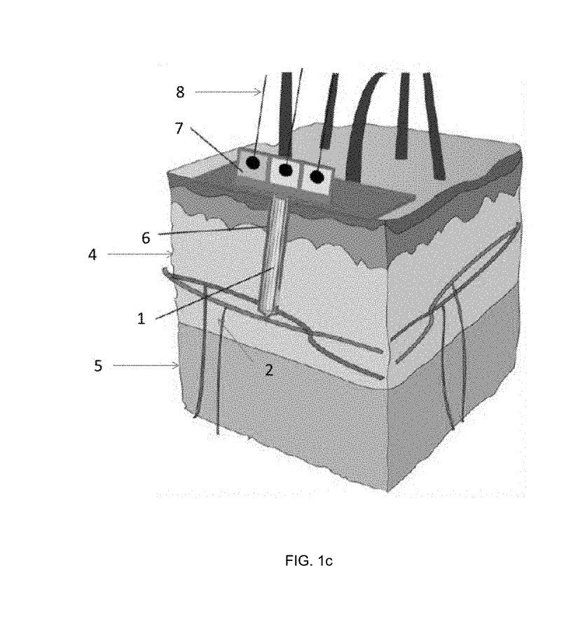

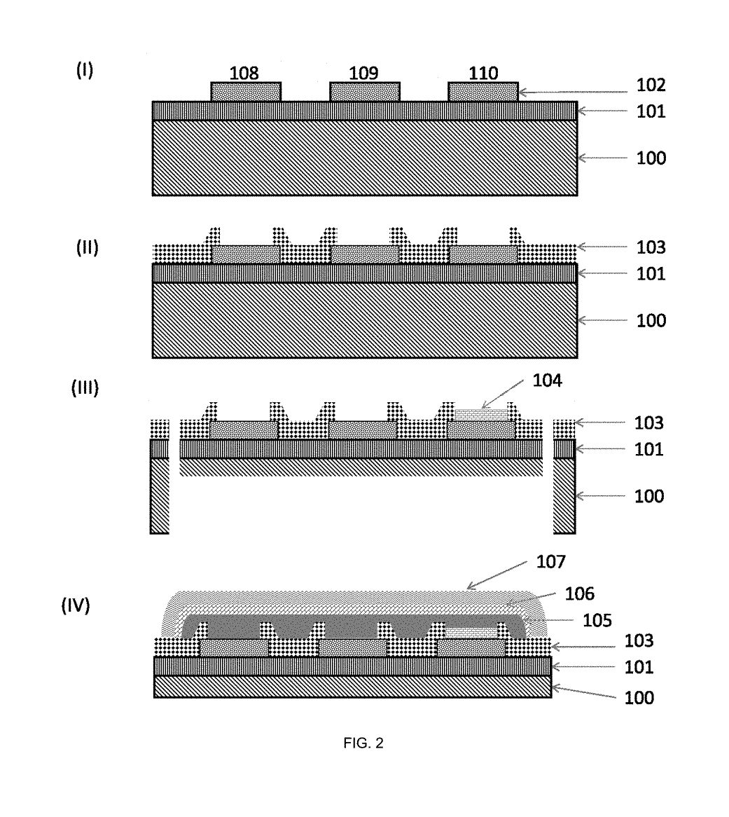

According to the FIG. 6, the WE may be arranged at the distal part of the probe (the nearest to the terminal end of the probe), the CE may be arranged at the middle part of the probe and the RE may be arranged at the more proximal part of the probe (for example, from the surface of the skin). The sensor is designed is such manner that the (all or substantially all) electrodes may be (fully or substantially fully) inserted in the dermis of a patient as shown in the FIG. 1b.

In other potential embodiment disclosed by the FIG. 7, the electrodes may be arranged in the width (horizontally). In this case all electrodes are inserted at the same depth of the patient skin.

While there are clear medical advantages, as described in the introduction, the miniaturization of this kind of sensors intrinsically entails challenges in signal amplitude, stability, choice of the active materials and valid fabrication processes. In fact, the amperometric current generated in the sensor is in a first approximation proportional to the active surface of the working electrode, and the reference electrode stability is decreased with the size reduction as well. Only the noise level, the electrode polarization and the ohmic losses are expected to be lowered and favor the performance when shrinking the device and placing the three electrodes on the same substrate. Despite the low currents involved in an amperometric sensor of this size, a three electrode setup has been chosen to guarantee better stability of the IrOx electrode even at high glucose concentrations; A significant drift in the current response at glucose concentrations above 200 mg/dL was previously reported in the case of a basic two-electrode configuration.

### Chemicals and Instrumentation

For information, Phosphate buffered saline (PBS, pH 7.4), glucose oxidase (Aspergillus niger, type VII), bovine serum albumin (BSA), glutaraldehyde grade I (GA, 25%), d-(+)-glucose, Nafion® 117 (5 wt % solution in a mixture of lower aliphatic alcohol and water), polyurethane Selectophore™ (PU), tetrahydrofuran (THF), N,N-dimethylformamide (DMF), L-ascorbic acid (AA) and uric acid (UA) were purchased from Sigma-Aldrich and used as received, if not specified otherwise.

The quality of the deposited materials and of the polymeric membranes have been controlled through scanning electron microscopy (SEM) and optical microscopy respectively, while the thickness of the membranes has been measured using a mechanical profiler. Electrochemical measurements were performed using a sub-picoampere resolution potentiostat (DY2011, Digi-Ivy, Inc.). A commercial 3M Ag/AgCl reference electrode (REF321, Radiometer Analytical) was used as a reference during electrode preparation. All potentials are referred to a saturated Ag/AgCl reference electrode, if not specified otherwise. For simplicity and to limit evaporation of the testing solutions during long term characterization, all experiments were performed at 26° C., unless stated otherwise.

### Example of Device Fabrication

### General Manufacturing Process of the Invention

Focused now to the FIG. 2, the present document further discloses a manufacturing process of a sensor disclosed by the invention. The sensor may comprise a substrate, for example a nonconductive substrate such a wafer having a silicon layer 100 and/or a silicon dioxide layer 101.

The step (I) may comprise the step of depositing at least one conducting layer (preferentially on the wafer, more preferentially on the silicon dioxide 101) defining a first electrode 108, a second electrode 109 and a third electrode 110. Preferentially, three distinct layer are deposited in order to define the first electrode, the second electrode and the third electrode, nevertheless, only one conducting layer may be deposited and patterned (for example via a mask or not) to define the three electrodes

The first electrode 108 may act as a working electrode, the second electrode 109 may act as a counter electrode and the third electrode 110 may act as a reference electrode. The conducting layer may be a platinum layer.

The step (II) may comprise the step of depositing a passivation layer for the interconnections (also called an insulation layer 103) at least between two conducting layers. Said insulation layer may be a silicon nitride. This layer insulates the metal interconnection from the measurement solution during operation. Additionally, it may anchors the electrodes' edges, avoiding liquid contact to the adhesion layers beneath.

The step (III) may comprise the step of depositing an iridium oxide layer 104 on the third electrode 110. This step may further comprise the step of cutting the sensor from the (initial) wafer (or at least before the step (IV). Step III (Iridium deposition) and step II (silicon nitride deposition) can be performed in reverse order without any change in the working principle.

The step (IV) may comprise the step of depositing at least one membrane on at least one of the three electrodes. At least one membrane may comprise linearity enhancing material which extends the linearity range of the sensor such as Polyurethane, Cellulose Acetate or other similar material. Additionally, at least one membrane may comprise permselective material which provides selectivity such as Nafion or Polypyrrole or other similar material.

Preferentially, the at least one membrane may comprise glucose oxidase, polyurethane and/or Nafion. The Nafion membrane and the polyurethane membrane may be optional for sensing glucose. The addition of Nafion provides better selectivity and PU enhances linearity, but these materials may be change by other similar materials. By changing enzyme (glucose oxidase), the sensor may measure any substance having a related oxidase enzyme (e.g. lactate, glutamate, etc.). In other embodiment, the membrane does not comprise any enzyme in order to obtain an electrochemical enzyme-free sensor or the membrane may comprise fluorescence-based material.

The electrode size range may be between 1 and 1000 μm at least in one dimension, preferentially between 100 and 400 μm at least in one dimension and between 30 and 70 μm at least in one dimension (for example an other dimension from the previous).

The inter-electrode distance may be in the range 1 and 1000 μm, preferentially 5 and 50 μm.

### Wafer Level Processing

The microsensors may be produced using photolithography and microfabrication technologies on a 100 mm <100> silicon substrate (for example). A 1-μm thick silicon dioxide layer may be thermally grown on the Si wafer to electrically insulate the electrodes from the substrate. 150 nm of platinum were deposited using electron-beam evaporation, over a 20 nm chromium adhesion layer, and patterned via lift-off in order to define electrodes, contacts and interconnections on the different chips. A 200 nm silicon nitride (Si₃N₄) layer may be then deposited using plasma enhanced chemical vapor deposition (PECVD). Openings, corresponding to contact pads and active sites of the microelectrodes may be defined using photolithography and wet etched in a buffered hydrofluoric acid (BHF) 7:1 aqueous solution. Finally, 80 nm of iridium may be selectively deposited using electron-beam evaporation on top of only the Q-RE microelectrode sites, over a 20 nm thick titanium adhesion layer, through a second lift-off step. The fabrication process is illustrated in FIG. 2.

### Electrode Functionalization

To obtain the desired IrOx layer on top of the evaporated iridium at the Q-RE site, cyclic voltammetry may be used. It is known that cycling the potential of iridium between oxygen and hydrogen evolution extremes leads to the creation of a thin oxide layer on the surface. The process may be carried out in a 0.1 M PBS solution, pre-deaerated for 10 minutes by nitrogen bubbling. The potential of the electrode may be then cycled 30 times between the extremes −0.75 V and +0.95 V, with a sweep rate of 200 mV/s, using a separate platinum strip as counter electrode and the Ag/AgCl electrode as reference.

Subsequently, at least one additional membrane may be deposited on the sensing region, in order to guarantee the desired performances in term of specificity, linearity and selectivity respectively. For example, the document disclose three membrane which may be placed by drop casting and may be let dry for 45 min each before further processing. A first membrane, for example, which embeds the enzyme and is responsible for the H₂O₂generation from glucose molecules, may be deposited using two aqueous solutions, consisting of (for example) 100 nL of 3 wt % GOx and 3 wt % BSA, and 100 nL of 2 wt % glutaraldehyde respectively, with the latter acting as crosslinker. A second membrane may consist of a permselective membrane, made from (for example) 800 nL of 3 wt % PU in a solution of 97% THF and 3% DMF. Due to the different diffusivity of glucose and oxygen through the PU layer, the linear range can been extended to guarantee sufficient resolution over the entire physiological glucose concentration range and avoid saturation. A third membrane, which may be semipermeable layer, may be made from a solution of 5 wt % Nafion (for example) and may be used to exclude anionic electroactive substrates, such as UA and AA, present in the IF. Nafion coatings have previously been shown to be permeable and allow transport of cations and neutral species, as well as supporting electrolytes, while rejecting negatively charged substrates. In addition to the increase in selectivity, this layer may protect the reference electrode from fouling and, hence, increases the stability of the sensor over time. Additionally, Nafion encapsulation has shown a good degree of biocompatibility, and is therefore a suitable interface for long term implantation use. After preparation, the sensors may be stored dry and thoroughly washed with DI water before being used for measurements.

### Results and Discussion

The present invention will be better understood at the light of the following paragraphs which contains a non-limiting example of an experimental test.

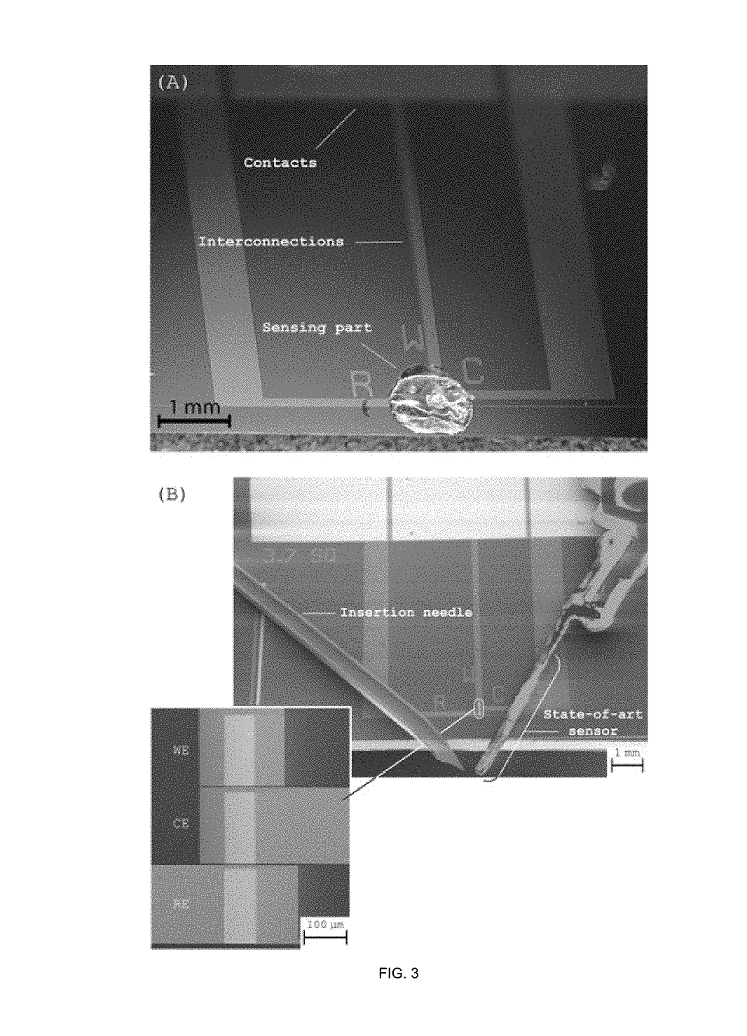

FIG. 3(A) shows an optical image of the top of an experimental chip (based on the invention and used for the test), after membrane casting. For the ease of manipulation and in-vitro testing, the contact portion of the chip has been kept macroscopic (around 1 cm² overall), while the targeted miniaturization of the probe-arranged active region of the sensor has been successfully attained. A reduction in total surface of the sensing portion of more than nine-fold has been achieved with respect to previous work and a nearly forty-fold decrease has been obtained if compared to state-of-the-art products, e.g. Abbott's Freestyle™ systems. The latter commercial sensor, together with its insertion needle, is shown in FIG. 3(B) in comparison to our bare microfabricated sensor. The developed sensor, with a total sensing portion footprint of 530×70 pmt, is small enough to potentially allow direct continuous glucose monitoring in the dermal region. The small size may also improve the durability and reliability of transdermal sensors themselves, due to the consequent limitation of detrimental FBR effects caused by their insertion and prolonged implantation. These advantages are combined with the fact that such a miniaturized device can result in virtually painless insertion into the skin, since its small size reduces the risk of encountering or stimulating a nerve, and hence of producing a painful sensation.

### Pseudo-Reference Electrode Stability

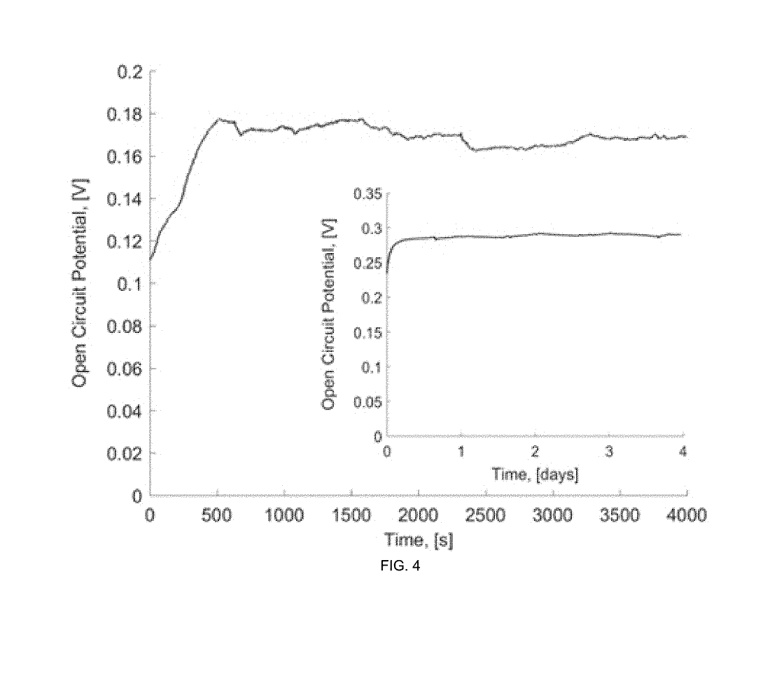

A fundamental requirement for the on-chip integrated Q-RE is to maintain, under standard physiological conditions, a constant potential over time, to provide a stable reference to the system. To assess the stability of the IrOx electrode, the open circuit potential (OCP) of fabricated iridium oxide pseudo-reference electrodes was measured in PBS, using a platinum strip as CE and the Ag/AgCl electrode as a reference. Long term stability of the open circuit potential of the produced Q-RE was evaluated over a four-day period, and a typical OCP curve is shown in FIG. 4. The inset in FIG. 4 shows the short term OCP of the Q-RE used in the ensuing measurements, immediately after oxidation in a deaerated PBS solution.

The obtained results partially differ from an earlier reported behavior. In that study a radical change of the OCP was measured after 24 hours, due to hydration of the IrOx film, after which a stable plateau was reached and maintained for the following nine days. In our study the OCP of the IrOx electrodes never changed significantly over a four-day time frame A probable explanation of the different behavior seen in this study with respect to earlier results is the different technique used for the deposition of the iridium layer and its oxidation, namely e-beam evaporation and successive electrochemical oxidation in our case, instead of direct IrOx electrodeposition. Nevertheless, the standard deviation is comparable (around ±10 mV), combined with a negligible drift over time in both cases. If the inter-electrode OCP variation is smaller than few tens of mV, it is then sufficient to properly choose the applied bias voltage, i.e. a voltage sufficient to always guarantee H₂O₂ oxidation without activating other unwanted reactions, in order to always guarantee correct functioning of the sensor. As an alternative for improved accuracy, sensor calibration can be performed, as currently required by several commercial CGMS. The different absolute values of OCP shown in FIG. 4, 170 mV and 295 mV respectively, can be partially explained by a previously reported study, where IrOx electrodes showed different standard potential in deaerated and oxygen-containing solutions. Preferentially, the short term OCP measurements were performed immediately after CV in a previously N₂-bubbled PBS solution, while the long term OCP studies (inset in FIG. 4) were performed in freshly made PBS solutions. However, further investigation is needed to better understand the iridium oxidation process and to characterize, and possibly optimize, the thickness of the created oxide layer, which influences the long-term stability.

Additionally, the oxidation of iridium can occur in different states, depending on the activation conditions, such as oxidizing solution and CV parameters. This influence on the OCP and the electrode stability have not been investigated in this work, even though a better understanding might improve durability and reproducibility of the produced electrodes.

### Amperometric Sensor Response

Plot 5(A) reports the amperometric glucose detection as a function of time, as well as the response to interferents (UA and AA) of the sensor. The sensing portion of the chip was immersed in the measurement solution, consisting of magnetically stirred 0.1 M PBS, and allowed to stabilize for a few minutes, before applying a bias of +0.5 V to the WE with respect to the integrated Q-RE. When the background current reached a stable value, first uric acid and then ascorbic acid were added to the solution, resulting in (with a final concentration of 100 μM each), in order to test the selectivity of the sensor. Afterwards, aliquots of concentrated glucose solution have been regularly added to raise the overall glucose concentration from 0 to 500 mg/dL, in steps of 50 mg/dL. The entire measurement lasted for more than eight hours, in order to show the stability of the reference electrode over prolonged use and to simulate the intended application of the device. Plot 5(B) shows, for the same measurement, the current as a function of the glucose concentration and demonstrates good linearity up to 300 mg/dL (and an excellent linearity up to 200 mg/dL) and adequate resolution over the full range of interest.

The goal of this study was to investigate miniaturization beyond previously published devices, and it is interesting to note that the obtained sensing performances compare positively to both current commercial and research-level systems, although some of the earlier sensors have the clear advantage of having been tested in-vivo. The sensitivity of the sensor is 1.51 nA/mM in the linear range. In order for this value to be compared to larger electrodes from previous work, the sensitivity can also be normalized, dividing its value by the WE area, to 12.7 pA·mM⁻¹·cm⁻². The obtained sensitivity is directly comparable to 1.04 nA/mM and compares favorably, in terms of normalized sensitivity, to 0.7 pA·mM⁻¹·cm⁻², 2.4 pA·mM⁻¹·cm⁻², 2.5 pA·mM⁻¹·cm⁻², 2 pA·mM⁻¹·cm⁻², and 3.3 pA·mM⁻¹·cm⁻² which have been claimed by prior studies.

Current International Organization for Standardization guidelines (ISO 15197:2013) require a resolution of ±20% at glucose concentrations above 100 mg/dL, justified by the fact that an error up to 20% is very unlikely to lead to a wrong medical decision. In this regard, the noise would limit the measurement resolution to ±5%; however, its influence can be completely eliminated by a simple averaging operation over time. Therefore, the main source of error, together with Q-RE drift, repeatability and reproducibility, is provided by the cross-sensitivity to endogenous interferents, which is hereafter analyzed in a worst-case scenario. At 26° C., the sensor exhibits adequate rejection of electroactive interfering species: the effect on the measurement current is limited to 1.32 pA/μM for UA and 1.69 pA/μM for AA. Moreover, the selectivity and functionality of the sensor have been tested in a PBS solution at 34° C., in order to better simulate the temperature in the dermal region, which is the targeted sensing location. This provides a more realistic characterization, since at higher temperature the reactivity of all involved species, including GOx, increases. Aliquots of ascorbic acid, uric acid and glucose have been added to the solution at different increasing concentrations while recording the amperometric response. In particular, plot 5(C) shows a detailed response of the sensor to AA and UA at three different concentrations: low standard concentration (AA at 0.6 mg/dL, UA at 2 mg/dL), high standard concentration (also defined as therapeutic levels, AA at 0.9 mg/dL, UA at 5 mg/dL), and toxic concentration (AA at 2 mg/dL, UA at 10 mg/dL). The current generated by the lowest levels can be considered as a minimum fixed offset of 0.34 nA with respect to the background current, which in this case equals 0.045 nA. On the other hand, fluctuations at higher interferent concentrations must be considered as errors. At therapeutic levels, the maximum introduced error is lower than 2.5%, while the maximum effect on the current is 8.0% at high toxic concentrations, proving sufficient glucose selectivity even under extreme testing conditions.

Finally, the average response time, calculated between the instant of glucose addition and the time at which the current reaches 90% of the plateau value, is around 300 s, which corresponds to a reaction time of 10 mg·dl⁻¹·min⁻¹. The rate of glucose change in humans is typically lower than 3 mg·dl¹.min⁻¹, and therefore the sensor would also manage to successfully track also extreme glycaemia variations.

The linearity range, which is 0 to 200 mg/dL for the sensor shown, can be enhanced by simply increasing the thickness of the permselective PU layer, at the expense of the sensitivity; an increased thickness would provide a safer margin in case of hypoxemia, as previously shown. In particular, an optimization of the trade-off between sensitivity and linearity should be performed, at a later stage when testing in-vivo, to balance performance and reliability under extreme medical conditions. While the described working principle has been verified on multiple sensors (n>10), and has been tested also with different electrode sizes and geometries, the manual drop casting deposition inevitably results in membrane thickness variations and, thus, in variation of amperometric response parameters, i.e. sensitivity and linearity range. The average thickness of the three polymeric membranes and their variability were measured with a mechanical profiler. The results are reported in Table 1.

TABLE 1

| Deposited membranes | Thickness [μm] | Variation [μm] |
|---|---|---|
| Layer 1: GOx-BSA-GA | 2.25 | ±1 |
| Layer 2: Polyurethane | 19.5 | ±4.5 |
| Layer 3: Nation | 1 | ±0.3 |

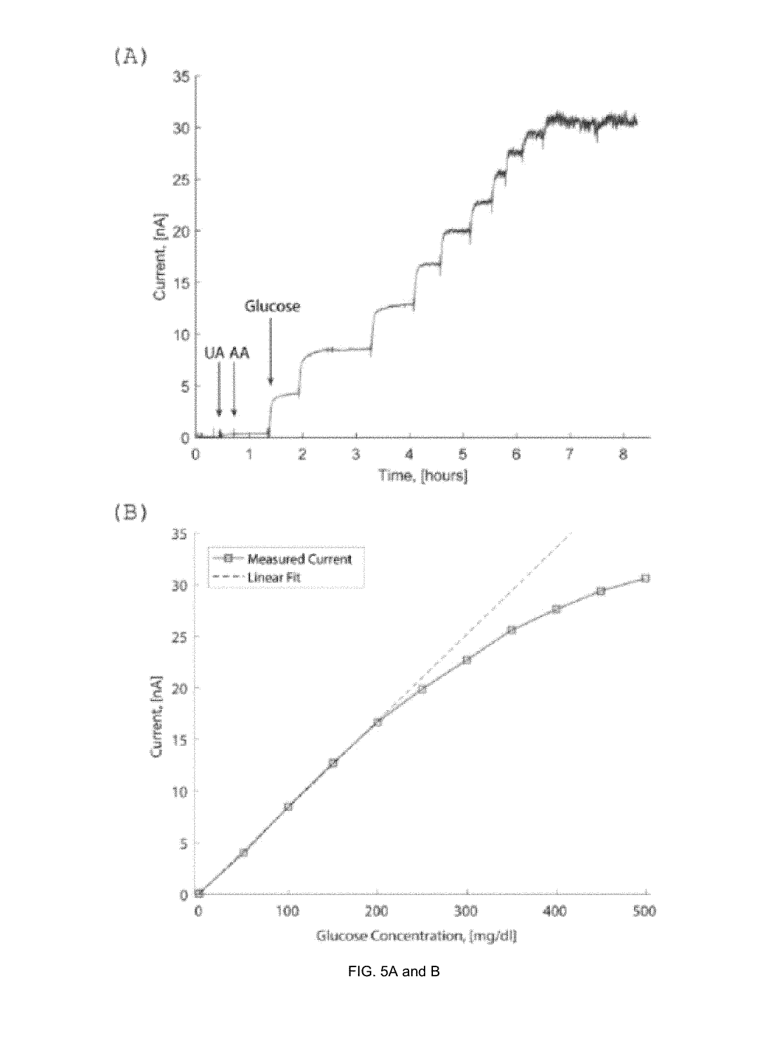

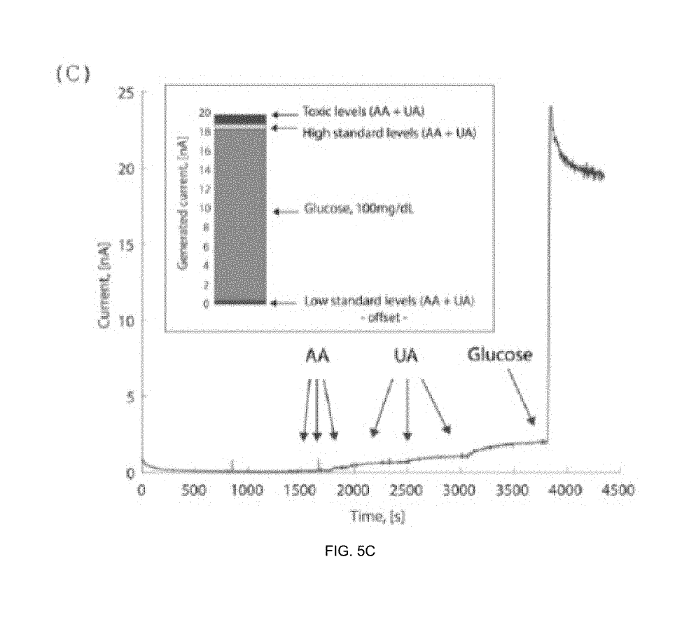

These variations were in fact found to directly affect the amperometric response parameters during characterization, in particular the sensitivity and the linearity range. For example, the variation in the polyurethane layer thickness from 15 to 24 μm results in a 60% reduction in sensitivity, as well as in an extended linear range. This can also be seen when comparing the response from the sensors in FIGS. 5(A) and 5(C) which were prepared using the same procedure. The second sensor has a higher sensitivity, which is partially due to the increased temperature (26° C. to 34° C.), but also due to a slightly thinner PU layer. On the other hand, this resulted in a reduced linearity range which is in accordance with the previously discussed trade-off between sensitivity and linearity. A proven and ideal solution to improve the reproducibility of the membrane deposition process would involve the use of more controllable and scalable membrane deposition techniques, e.g. inkjet printing or automated CNC-based liquid dispensing. In fact, a typical inkjet drop is typical between 1-10 pL, which translates to a thickness resolution in the order of 100 nm for the previously mentioned materials. This would result in negligible inter-sensor membrane thickness variations and, hence, likely more reproducible results. Additionally, this would also allow the enzyme layer to be placed on top of only the working electrode, thereby improving sensor performance.

Nevertheless, despite the non-ideal deposition conditions, the sensors were demonstrated to fulfill the initial requirements in terms of performance and overall size, and thus demonstrated the prospect of a significant possibility of down scaling possibility for amperometric continuous glucose monitoring systems.

Further sensor miniaturization has also been investigated; for example, a microsensor having a footprint of 385×60 um² and a thicker PU membrane guaranteeing excellent linearity up to glucose concentrations above 600 mg/dL has been realized and tested. At this size and conditions the signal is still clearly detectable; however, the sensitivity drops dramatically and reduces to 0.03 nA/mM (data not shown).

Future challenges for this work include integration of the described electrodes on mechanically resistant probes allowing in-vivo intradermal measurement of the glucose concentration in interstitial fluid. In this regard, additional precautions may be needed in order to guarantee proper functionality in presence of other exogenous interferents (e.g. acetaminophen), or in case of extreme hypoxemia, as previously discussed. Sufficient robustness (or protection) of the polymeric coatings has also to be provided for a safe insertion.

### Example of Sensing Device

### Device Design

As explained in the prior art part, a micro sensor may be brittle and thus the insertion step of such sensor into the patient skin may be difficile or impossible. Thus, the present document further disclosed a sensing device comprising a microneedle and a sensor arranged into the needle.

Preferentially, the microneedle is not used for sensing but the microneedle is used for protecting the sensor. Thus the microneedle may comprise a non-conductive body or at least an insulating layer on a wall (internal and/or external wall) (for example an oxidation layer such as silicon dioxide or other).

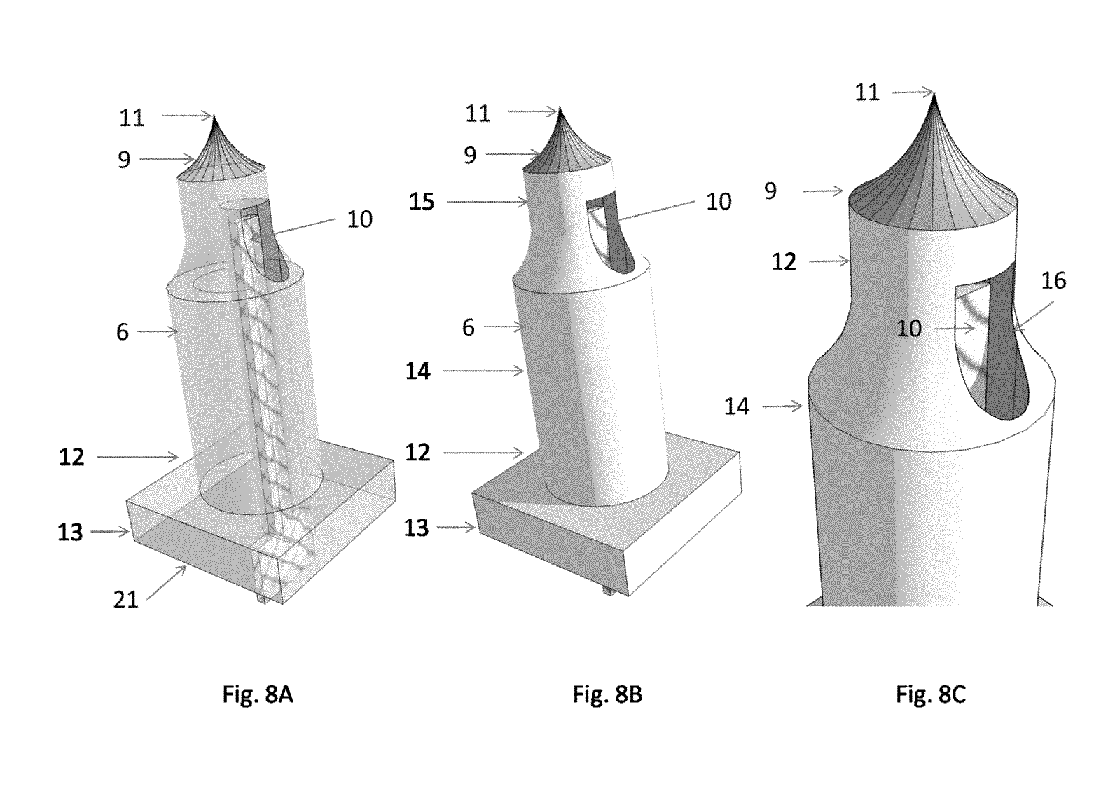

The FIGS. 8 A, B and C show an example of such sensing device. The FIG. 8A shows a transparent view of the needle.

In one embodiment, the sensing device comprises:
an hollow microneedle 6 having:
   a longitudinal body 14 which extends from a base 12 to a distal end 11 (which comprises preferentially a sharp tip),
   at least one internal wall defining an internal cavity, and
   an opening configured to access the cavity from the exterior of the microneedle; and
a sensor arranged at least partially into the cavity or into the microneedle.

In order to protection the sensor, the microneedle may further comprise a hat 9 arranged at the distal end configured to cover at least partially the opening or the sensor.

The cavity may define a vertical through hole which extends from the backside 21 of the microneedle to the distal end of the opening 16. In this case, a micro sensor may be inserted from the backside of the microneedle.

The microneedle (or a part of the microneedle) may comprise (or made of) a silicon material.

After the insertion, the microneedle may be less important or bring no specific feature, thus, all or a part of the microneedle may comprise (or made of) a dissolvable material. In this case, the microneedle is dissolved into the patient body after the insertion. For example, just the hat 9 or a distal end part of the microneedle may comprise a dissolvable material. Thus, after insertion, the hat 9 or the distal end of the microneedle is dissolved into the patient body. In these cases, the micro needle may not comprise any side opening 16.

Preferentially, the sensing device comprises a single microneedle in which a sensor (for example a full electrochemical cell) is arranged for example into a (single) cavity or channel or through hole. Thus, in this case, all electrodes of the sensor (for example, the RE, WE and the CE) are arranged into the single microneedle for example into the cavity or channel or through hole. The placement of the three electrodes on the same probe allows for reduction in the noise level, and reduction of electrode polarization and ohmic losses (due to the ionic resistance of the skin which leads to increased overpotential and energy losses).

In other cases, as described below, a microneedle may comprise several cavities or channels or through holes, and in this case, each or several electrode may be arranged in a dedicated cavity of the microneedle.

Preferentially the opening 16 is arranged at the head 15 of the microneedle. If the electrodes are arranged vertically, the opening may be configured in such manner that at least one electrode faces the opening 16 and the body fluid may flow through the closed cavity (for example by capillarity).

Preferentially, WE is located as close as possible to the opening for measurement delay minimization. As described below, the opening may be elongated in such a manner at least two or more electrodes face the opening 16.

Preferentially, the opening 16 is configured to extend (when inserted into the patient body) through or to reach a determined measurement area of the patient, for example a determined depth of a patient skin, such the dermal layer of the patient skin.

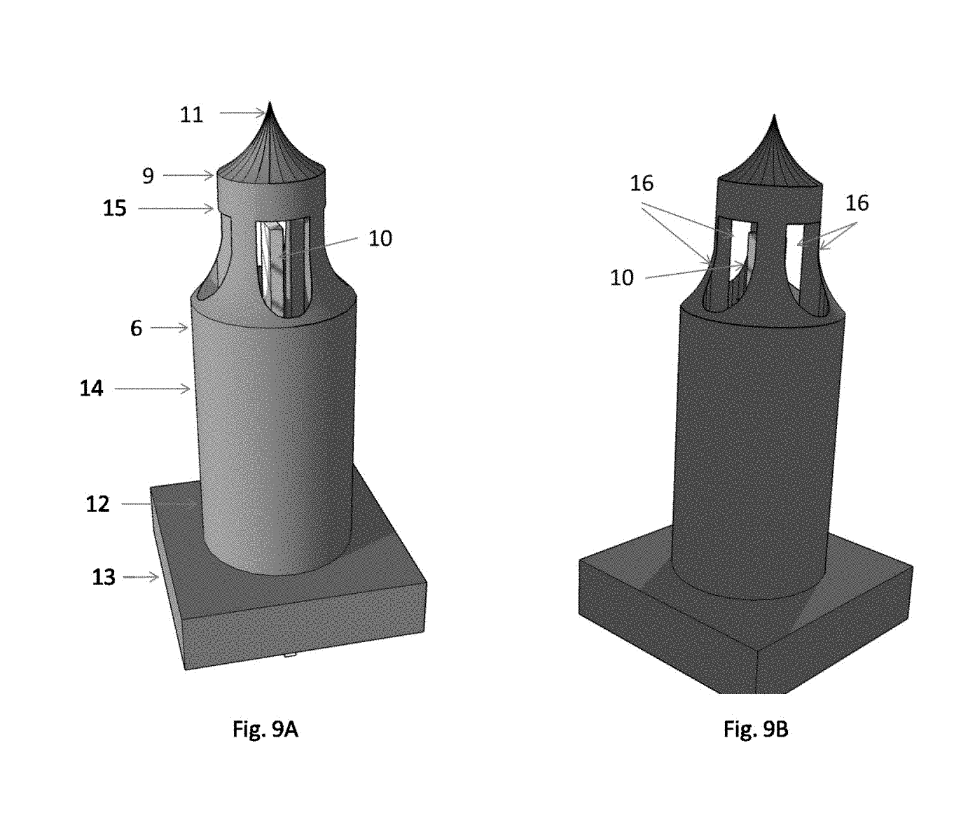

Focused on the FIG. 9, the microneedle comprises several openings 16 which are in fluidic communication with the sensor 10 or the cavity.

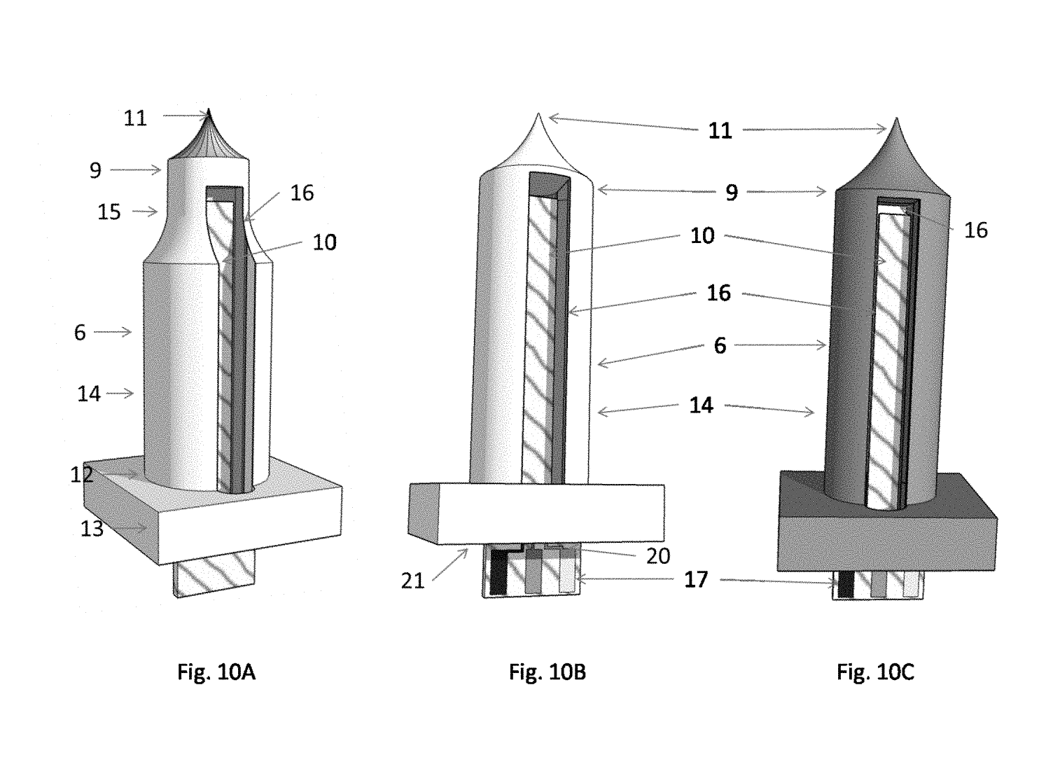

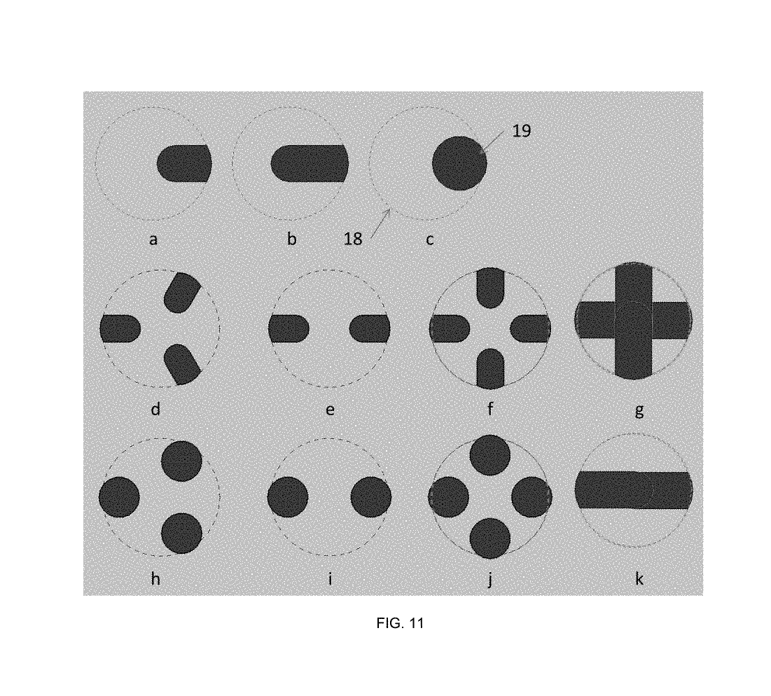

The electrical contacts 17 of the sensor 10 may be arranged in such manner that it extends outside of the microneedle as shown in the FIG. 10b.

Focused now on the FIG. 10, the sensing device comprises:
A microneedle 6 having:
   a longitudinal body 14 which extends from a base 12 to a distal end 11, and
   a (substantial) side opening 16; and
a sensor 10 arranged at least partially into the side opening; wherein the side opening 16 extends along of a determined length of the longitudinal body such that the first side opening reaches a determined depth of the patient skin when the microneedle is inserted into the patient skin.

The side opening may extend at least along 25% of a length of the longitudinal body or at least along 50% (or more for example 80-90-100%) of a length of the longitudinal body.

The side opening may extend at least along 25% of a length of a sensing part of the sensor or at least along 50% (or more for example 80-90-100%), such that the sensing part of the sensor contacts the interstitial fluid in a determined depth.

As disclosed by the FIG. 10C, the microneedle may further comprise an additional side opening which may be arranged opposite to the first side opening.

The support of the microneedle may be used to limit the penetrating depth. And the length of the elongated body of the microneedle is preferentially less than 1 mm.

### Manufacturing Process

The present document further disclosed a manufacturing process of microneedle having a large lateral opening(s) (side opening(s)) of the microneedle and to ensure that the opening(s) is (are) located over the substantial full height of the microneedles (for example). The circulation of the fluid on the sensor will thus be greatly increased, which will make it possible to obtain a more efficient and successful sensor.

The FIG. 12 shows a potential process flow for microneedle adapted to receive a sensor. The process may comprise the steps of:
Providing a wafer (for example a silicon wafer);
Depositing a mask on backside of the wafer;
Patterning of the through holes on the backside;
Depositing a protection layer such as a silicon dioxide, at least into the through holes;
Depositing a mask on front side of the wafer,
Patterning of the microneedle on the frontside, for example a first isotropic etching which is configured to etch the silicon;
Patterning of the microneedle on the frontside, for example a first anisotropic etching which is configured to etch the silicon;
Finalisation of the microneedle on the frontside, for example a second isotropic etching which is configured to etch the silicon;

The process may further comprise the step of cleaning, final oxidation and dicing.

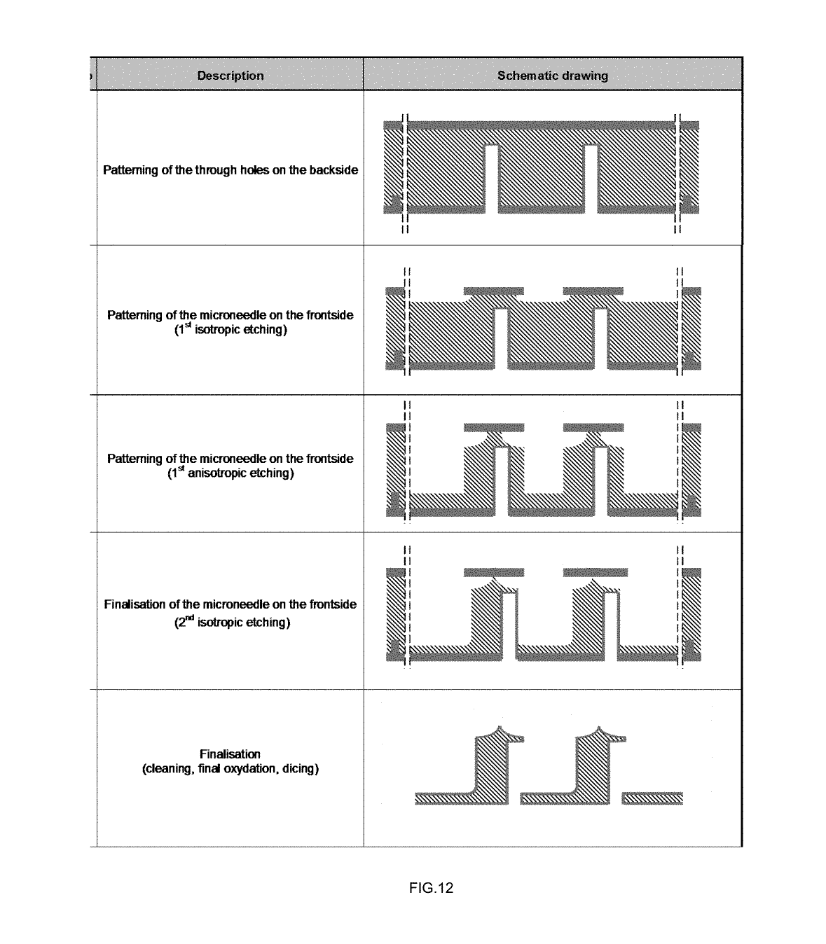

The FIG. 11 shows different shapes and sizes of the through holes on the backside. Several solutions may be conceivable:
a, b and c show a single through hole on the backside. a and b are oblong and c is a circle;
d and h show three distinct through holes on the backside. d is oblong and h is a circle;
e and i show two distinct through holes on the backside. e is oblong and i is a circle;
f and j show four distinct through holes on the backside. f is oblong and j is a circle;
g and k show a single through holes on the backside which allows several side opening communicating there between. These shapes allow flowing of the body fluid through the side opening.

1. A sensing device for sensing at least one analyte from a biological fluid of a patient, the sensing device comprising:
a hollow microneedle having:
a longitudinal body which extends from a base to a distal end,
at least one internal wall defining at least one internal cavity, and
at least one opening configured to access the at least one internal cavity from the exterior of the microneedle; and
a sensor device arranged at least partially into the hollow microneedle,
wherein the sensor device is a probe comprising at least two electrodes, arranged on the probe; wherein the probe comprises a longitudinal body and the at least two electrodes are arranged in the width or in the length of the longitudinal body of the probe.

2. The sensing device according to claim 1, wherein the at least two electrodes comprise a first electrode which is a working electrode and a second electrode which is a counter electrode, and wherein the electrode size is between 1 and 1000 μm at least in one dimension.

3. The sensing device according to claim 2, wherein the electrode size is between 100 and 400 μm at least in one dimension.

4. The sensing device according to claim 2, wherein the electrode size is between 30 and 70 μm at least in one dimension.

5. The sensing device according to claim 1, wherein the length of the probe is smaller than 1 mm, the width of the probe is smaller than 100 μm, and wherein the probe is adapted so the at least two electrodes reach determined depth of a patient skin.

6. The sensing device according to claim 1, wherein the inter-electrode distance is in the range of 1 and 100 μm.

7. The sensing device according to claim 1, wherein the inter-electrode distance is in the range of 5 and 50 μm.

8. The sensing device according to claim 1, wherein the second electrode is twice as large as the first electrode.

9. The sensing device according to claim 2, comprising a third electrode, preferably a pseudo reference electrode.

10. The sensing device according to claim 1, wherein the microneedle comprises a hat arranged at the distal end configured to cover at least partially the opening or the sensor device.

11. The sensing device according to claim 9, wherein the sensor device is made by a manufacturing process of the three electrodes, comprising a substrate, and wherein process comprises the steps of:
depositing at least one conducting layers defining a first electrode, a second electrode and a third electrode,
depositing an iridium oxide layer on the third electrode, and
depositing at least one membrane on at least one of the three electrodes.

12. The sensing device according to claim 11, wherein the deposited conducting layer is a platinum layer.

13. The sensing device according to claim 11, wherein at least one membrane comprises glucose oxidase.

14. The sensing device according to claim 11, wherein at least one membrane comprises at least one of: a) a linearity enhancing material which extends the linearity range of the sensor such as Polyurethane, Cellulose Acetate or other similar material; and b) a permselective material which provides selectivity such as Nafion or Polypyrrole or other similar material.

15. The sensing device according to claim 11, further comprising the step of depositing an insulation layer at least between two conducting layers, preferably the insulation layer is a silicon nitride.

16. The sensing device according to claim 11, wherein the substrate is nonconductive wafer comprising a silicon-layer and/or silicon dioxide layer.

17. The sensing device according to claim 1, comprising a first end having connecting contacts and a second end having the electrodes and intended to be inserted into the patient body

18. The sensing device according to claim 2, comprising the first electrode arranged in the terminal of the second end.
